(19) United States
(12) Patent Application Publication
(10) Pub. No.: US 2009/0292309 A1
(43) Pub. Date: Nov. 26, 2009

(54) System and workflow for diagnosing and treating septum defects

(76) Inventor: Michael Maschke, Lonnerstadt (DE)

Correspondence Address: BRINKS HOFER GILSON & LIONE, P.O. BOX 10395, CHICAGO, IL, 60610, US

(21) Appl. No.: 12/154,053

(22) Filed: May 20, 2008

(51) Int. Cl. A61B 17/03; A61B 6/03; A61B 8/13

(52) U.S. Cl. 606/213; 128/898; 600/433; 600/439; 604/96.01

(57) ABSTRACT

A system, method and workflow is described that may be used for the treatment of a patient having a cardiac syndrome characterized by an unwanted aperture in a structure of the heart. Images of the patient are obtained with a C-arm X-ray device, and the images are used to determine the size and location of the aperture. A catheter is used to insert an occluder into the patient and emplace the occluder in the aperture so as to close the aperture. The occluder may be guided using one of fluoroscopic images from the C-arm X-ray device, acoustic or magnetic sensors. Diagnosis of the syndrome and the minimally interventional treatment may be performed by the same treatment suite.

### TECHNICAL FIELD

The present application may relate to a system, method and workflow for the diagnosis and treatment of cardiac defects.

### BACKGROUND

When the human fetus is connected to the maternal metabolism via the placenta in the uterus, the exchange of substances with the mother takes place via the umbilical cord, which contains two arteries and one vein. The vein supplies the fetal organism with oxygen and nutrients from the maternal circulation, while the two arteries carry oxygen-poor blood and unusable products of metabolism from the fetus back to the maternal circulation. So that balanced fetal circulation possible and, at the moment of birth, the full function of the pulmonary circulation will immediately be assured, the foramen ovale, a crescent-shaped slit, is present between overlapping points of the primary and secondary atrial septum. Blood flow is possible from the lower hollow vein into the left atrium and a direct connection exists between the venous and the arterial system (right-to-left shunt).

Immediately after the birth, both the blood flow in the pulmonary circulation and the pressure in the left atrium rise. In most cases, this causes the foramen ovale to be closed by the valve-like action of the primary atrial septum and to remain closed as long as the pressure in the left atrium is greater than in the right atrium. However, in one-fourth of the adult population of Central Europe, for example, the foramen ovale does not close, or is not completely closed. Medically this is termed a persistent (or patent) foramen ovale (PFO).

Atrial Septum Defect (ASD) is a congenital hole in the septum between the atria. This defect in the atrial septum leads to a left-to-right shunt and, thus, to pulmonary inundation. Small ASDs exhibit a tendency to close spontaneously in the first year of life. However, depending on the size and location of the defect, closure by means of a cardiac catheter or surgery may be necessary.

Ventricular Septal Defect (VSD) is the most common congenital heart defect: a hole in the septum between the ventricles of the heart. However, a VSD may also occur as a consequence of myocardial infarction. Since the pressure in the left chamber is higher than in the right chamber, each time the heart beats, blood flows from the left chamber to the right chamber of the heart (left-to-right shunt) and from there into the lungs, causing pulmonary inundation. The larger the defect, the more blood flows through and, hence, the more serious are the consequences.

VSD and ASD make up half of all of the congenital defects of the heart.

In the fetal circulation, the ductus arteriosis is a normal connection between the pulmonary artery and the aorta. A few days after birth, the ductus closes spontaneously. If closure does not happen within a few weeks after birth, the situation is called a persistent ductus arteriosis (PDA). Children with a small PDA have no symptoms; if the PDA is large, an increased vulnerability to infection and even cardiac insufficiency can occur. Since all children with PDA have an increased risk for the occurrence of cardiovalvulitis, a ductus arteriosis that persists beyond the second year of life needs to be closed. If the ductus is large, with an increased lung flow, the aperture should be closed even earlier in life.

In patients with atrial fibrillation, thrombi that can lead to a stroke can be released through the left atrial appendage (LAA) a structure that that is superfluous to normal cardiac function. Observations of patients in whom the left atrial appendage was closed during the course of mitral valve surgery have shown that the risk of stroke in these patients can be reduced over the long term.

Correction of the aforementioned cardiac defects formerly required open-heart surgery with support from a heart-lung machine, during which the openings in the heart are surgically closed. This invasive procedure was first performed in the 1950s, and involved a high risk to the patient. However, for some years now, methods have existed for treating the above-mentioned syndromes using minimally invasive techniques using “occluders”, so as to close the undesirable openings in the heart structure.

Occluders are installed by means of minimally invasive cardiac catheter technology, typically by transvenous access. Such catheter-based techniques are distinguished by ease of manipulation of the occluder, self-centering of the occluder in the shunt and, if needed, retrievability of the occluder before detachment from the catheter guide wire. Occluders are available from, for example, AGA Medical (Plymouth, Minn.) suitable for minimally invasive interventional therapy of the heart.

Depending on the specific syndrome to be treated, and the size of the defect, occluders can be furnished in various sizes in terms of the screen and waist diameter. The occluder devices are often based on the use of a nitinol wire cloth which, in the shape in which it is deployed, looks somewhat like the shape of a yo-yo: that is, the occluder has two retention screens and a waist portion disposed between the screens.

Because of the elastic properties of the nitinol material used, occluders can be narrowed after forming, by stretching the cloth in such a way that the occluder can be introduced into the septal defect to be closed using a catheter, or into the shunt of the septal defect that is to be closed.

After leaving the catheter, the two retention screens are deployed automatically and, after detachment from the introduction wire, the screens assume a flattened form on both sides of the septum. The waist of the occluder centers itself automatically on the hole during the deployment of the screen in the shunt that is to be closed. Non-woven inlays in the retention screens reinforce the sealing-off of the bloodstream. The hub of the wire cloths, which can be a threaded coupling for connection to the introduction catheter, is located in the right atrium, while there is no hub protruding into the left atrium. Thus, occluders can thus be inserted to close off the septum flat on the left atrial side of the septal defect, and contribute to faster endothelialization.

Closure of the left atrial appendage (LAA) can also be done by catheter technology if, after the left atrium is punctured trans-septally, a closure system is introduced through a long trans-venous lock.

A disadvantage of the above techniques is that the devices are placed in the heart with the aid of radiological imaging, and during this procedure both the patient and the medical personnel are exposed to X-radiation. Typically, when using X-ray devices for visualization, either the occluder is readily visible, or the opening in the septum is readily visible, but not both at the same time due to dynamic range limitations. By injecting contrast agent, the opening can be visualized more clearly, but there are patients who react allergically to contrast agent. Also, because of the restricted view, there is the risk that the occluder will not be placed correctly.

EP 1 211 980 B1, “Surgical Scanner System”, discloses a system in which a computed tomography scanner can be moved over a patient for examination using a rolling device. This solution also makes it possible to examine soft tissue, such as cardiac tissue, but has the disadvantage that during the examination, there is no access to the patient. Moreover, only CT examinations with a high dose of radiation are possible.

### SUMMARY

A system for treating a heart syndrome is described, including an imaging modality, configured to produce CT-like 3 dimensional (3D) images or voxel data sets; and a catheter capable of introducing an occluder device into a heart of a patient. The occluder device is selected based on a measurement of a size of an aperture in the heart of the patient using the 3D images or voxel data sets.

In another aspect, a method of treating cardiovascular syndromes is disclosed, the method including, positioning a patient in a therapy unit; determining the size of an aperture between structures of the heart using a C-arm X-ray device; and, emplacing an occluder in the aperture using a catheter and guide wire.

In yet another aspect, a method of treating a patient includes, providing a patient treatment suite, the suite further comprising: a C-arm X-ray device, configured to produce CT-like 3 dimensional (3D) images or voxel data sets; and, a catheter suitable for introducing an occluder into a heart of the patient. The occluder is selected based on a measurement of a size of an aperture in the heart of the patient.

In a further aspect, a workflow for treating a patient having a septum defect, is described, the workflow including the steps of: placing the patient on a patient support in a treatment unit; inserting a catheter into the patient; obtaining images of a heart of the patient using a C-arm X-ray device capable of recording data suitable for the production of CT-like images. The size of an unwanted aperture in the heart is measured using an imaging modality and occluder is selected based on the measured aperture size. The occluder is introduced into the patient using a catheter having a guide wire, and emplaced. Subsequently, the steps of detaching the occluder from the guide wire; removing the catheter from the patient; and transporting the patient from the treatment unit are performed.

### BRIEF DESCRIPTION OF THE DRAWINGS

FIG. 1 is a block diagram showing the component equipment of a treatment unit for septum defects; and

FIG. 2 is a flow chart of the workflow for diagnosing and treating septum defects.

### DETAILED DESCRIPTION

Exemplary embodiments may be better understood with reference to the drawings. In the interest of clarity, not all the routine features of the implementations described herein are described. It will of course be appreciated that in the development of any such actual implementation, numerous implementation-specific decisions must be made to achieve a developer's specific goals, such as compliance with system and business related constraints, medical potocols and regulatory requirements, and that these constraints will vary from one implementation to another.

A “cardiac treatment unit”, “treatment unit”, or “therapy unit” in which the patient need not be transported from place-to-place between the individual steps in diagnosis and therapy is described. Such a therapy unit may include at least some of the following equipment types integrated as a platform for performing diagnosis and treatment of a patient: an imaging modality, which may be a C-arm X-ray unit capable of producing radiographic data for computed tomography (CT)-like (3D) and fluoroscopic (2D) images; and, one or more of: an image processor for at least one of soft tissue or angiographic image data obtained by the imaging modality; a sensor for detecting patient motion; a processor for motion correction of images for patient motions; a patient monitor, which may include a vital signs monitor and an EKG; an image fusion processor; a blood analysis device; a computer and interface for entering patient data; and a data interface with a local area network or a wide area network. Suitable image and data display devices such as flat panel video displays are also provided.

The combination of hardware and software to accomplish the tasks described herein may be termed a platform or “therapy unit”. The instructions for implementing processes of the platform may be provided on computer-readable storage media or memories, such as a cache, buffer, RAM, removable media, hard drive or other computer readable storage media. Computer readable storage media include various types of volatile and nonvolatile storage media. The functions, acts or tasks illustrated or described herein may be executed in response to one or more sets of instructions stored in or on computer readable storage media. The functions, acts or tasks may be independent of the particular type of instruction set, storage media, processor or processing strategy and may be performed by software, hardware, integrated circuits, firmware, micro code and the like, operating alone or in combination. Some aspects of the functions, acts, or tasks may be performed by dedicated hardware, or manually by an operator.

In an embodiment, the instructions may be stored on a removable media device for reading by local or remote systems. In other embodiments, the instructions may be stored in a remote location for transfer through a computer network, a local or wide area network, by wireless techniques, or over telephone lines. In yet other embodiments, the instructions are stored within a given computer, system, or device.

Where the term “data network”, “web” or “Internet” is used, the intent is to describe an internetworking environment, including both local and wide area networks, where defined transmission protocols are used to facilitate communications between diverse, possibly geographically dispersed, entities. An example of such an environment is the world-wide-web (WWW) and the use of the TCP/IP data packet protocol, and the use of Ethernet or other known or later developed hardware and software protocols for some of the data paths. Often the internetworking environment is provided, in whole or in part, as an attribute of the facility in which the platform is located.

Communications between the devices, systems and applications may be by the use of either wired or wireless connections. Wireless communication may include, audio, radio, lightwave or other technique not requiring a physical connection between a transmitting device and a corresponding receiving device. While the communication may be described as being from a transmitter to a receiver, this does not exclude the reverse path, and a wireless communications device may include both transmitting and receiving functions. Such wireless communication may be performed by electronic devices capable of modulating data as signals on carrier waves for transmission, and receiving and demodulating such signals to recover the data. The devices may be compatible with an industry standard protocol such as IEEE 802.11b/g, or other protocols that exist, or may be developed.

The term “care path”, “clinical care plan”, “workflow” or similar terms as used herein refers to a workflow or method in a medical context that includes a number of worksteps associated with the diagnosis and/or treatment of an illness. For example, typical worksteps within a care plan may include admission, screening, diagnostic testing, therapy, physical examinations, operations, ambulance care, out-patient care, in-patient care, oncology related care, and other steps. Worksteps may include a sequence of process steps, the use of specified treatment or diagnostic equipment, medical supplies, such as contrast agents, occluders, stents, drugs, medical appliances, transportation of the patient, performing medical procedures requiring at least one of non-invasive, minimally invasive or invasive aspects, and the like.

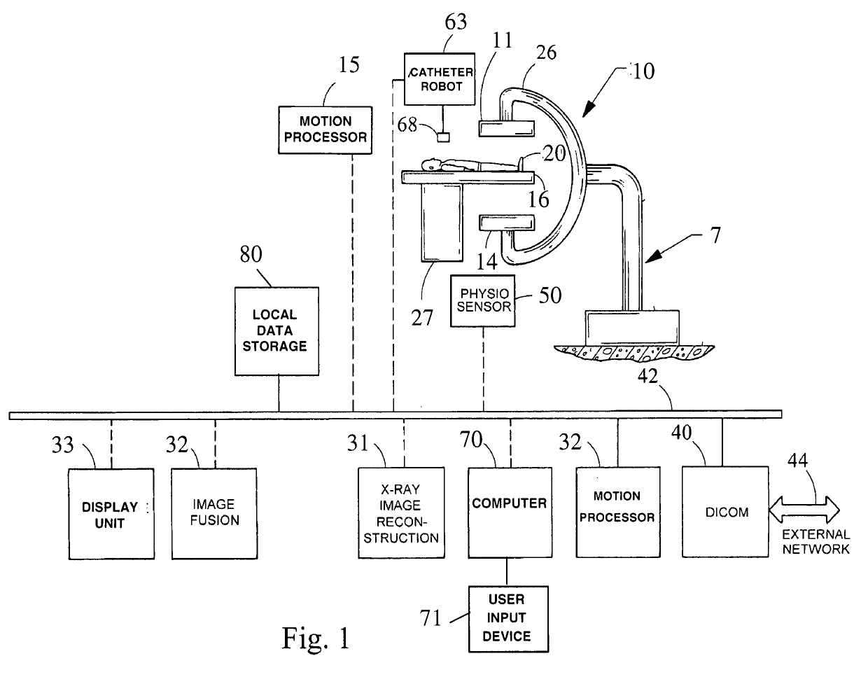

Minimally invasive therapies, especially cardiological procedures, including diagnosis and treatment of cardiac septum syndromes may be performed by a therapy unit that may include an X-ray system with which CT-like soft-part tissue images can be produced. FIG. 1 shows a block diagram of such a therapy unit.

The C-arm X-ray device 10 is representative of imaging modalities which may be used. In an aspect, the device may include at least one multi-axial robot on which the C-arm 26 having a radiation source 11 and a detector 14 attached thereto are mounted, and the C-arm moved in arbitrary paths, usually in circles, ellipses or spirals, around, or partially around the patient in order to make projection radiographic images. The C-arm X-ray device 10 may be rotated such that a sequence of projection X-ray images is obtained by an X-ray detector 14, which may be a flat panel solid state two-dimensional detector positioned on an opposite side of the patient 20 from the X-ray source 11. The X-ray detector may be amorphous Silicon (a-Si), amorphous Selenium (a-Se), PbI2, CdTe or HgI2 detectors, or the like, using direct detection or TFT technology, or indirect detectors as is known in the art, or may be subsequently developed, to provide high resolution, high-dynamic-range essentially real-time X-ray detection.

A C-arm X-ray device does not completely surround a patient, as does a conventional CT apparatus. Thus, the C-arm X-ray device provides more convenient access to the patient during interventional treatment, without moving the patient with respect to the imaging modality, or disconnecting monitoring and therapy equipment. The CT-like data and corresponding fluoroscopic data may be obtained with the same device. Images may reconstructed so as to form CT-like voxel data sets, and segmented by any technique of image or data processing processing for realizing computed tomographic (CT) images and representations thereof.

Other imaging modalities, such as extracorporeal acoustic imaging may also be used, either alone or in conjunction with the C-arm X-ray device.

Additional, different, or fewer devices may be provided in the therapy suite. The devices and functions shown are representative, but not inclusive. The individual units, devices, or functions may communicate with each other over cables, wires, or in a wireless manner, and the use of dashed lines for some of the connections shown in FIG. 1 is intended to suggest that alternative means of data and control connectivity may be used.

The C-arm X-ray radiographic unit and the associated image processing may be of the type described in US PG-Pub Application US2006/0120507, entitled “Angiographic X-ray Diagnostic Device for Rotational Angiography”, filed on Nov. 21, 2005, which is incorporated herein by reference. Such an apparatus may produce angiographic and soft-tissue tomographic images comparable to, for example, CT equipment, while permitting more convenient access to the patient for treatment procedures.

The sensor portions of the therapy unit may be located in a therapy room, and some or all of the signal and data processing and data display may also be located in the therapy room; however, some or all of the equipment and functionality, not directly associated with the sensing of the patient, may be remotely located. Such remote location of portions of the equipment may be facilitated by high-speed data communications on local-area networks, wide-area networks, or the Internet. The therapy unit may thus be located remotely from the specialists making the diagnosis and for determining the appropriate course of treatment. Of course, the specialists may also be present with the patient in the treatment room.

A patient support table 16 may be used for some or all of the examination steps and thus may transfer the patient 20 between various sensors or otherwise position the patient 20. In an aspect, at least one multi-axis robot 27 can guide and/or hold the patient support 16. The robot 27 may have at least four degrees of freedom of motion, and may have six degrees of freedom of motion.

When an X-ray system such as the AXIOM Artis dTA DynaCT (available from Siemens AG, Erlangen, Germany) is used to perform computed tomography, with or without the administration of contrast agents, tomograpic (CT)-like images may be obtained during a diagnostic or interventional procedure. In such a use, image acquisition may take approximately 10 seconds with C-arm rotation through approximately 200 degrees.

A motion sensor 15 may be provided for detecting motion of the patient 20 during examination or treatment and taking the detected patient motion into account in a motion processor 32 in the image reconstruction processing unit 31. The motion sensor 15 may be a mathematical motion detector, for instance deriving from the image signals themselves, such as is described as in US Patent Application 2002/0163994, “In-Line Correction of Patient Motion in Three-Dimensional Positron Emission Tomography.” In another aspect, the motion sensor may be capacitive as in U.S. Pat. No. 6,661,240, “Method and System for Capacitive Motion Sensing and Position Control”; magnetic, as in EP 0993804, “Method and System for tracking an object; acoustical, as in EP 1034738; “Positioning Based on Ultrasound Emission”; or, optical, where the position of the patient may be detected by an optical or infrared camera, and by computational methods of pattern recognition.

In an aspect, the patient may be scanned with a laser beam. Patient displacements or shifts may be ascertained and corrected in an image fusion processing unit 32. Such processing units may be combined into a single processor, or be multiple processors, and a processing function may be represented as either hardware, software, or a combination thereof.

The motion sensor 15 may transmit data to the image fusion unit 32 or a separate motion processor 38 through a wired connection or in wireless form. Before the beginning of the treatment, the motion sensor 15 may be calibrated relative to the spatial coordinates of the various examination apparatus, and may be calibrated relative to the patient support table 16.

The function of eliminating motion artifacts may include motions that are due to breathing and the motion of the heart (for example, by “EKG gating”) and the blood vessels. A chest belt using suitable sensors may be used to ascertain the breathing amplitude and frequency, and initiate corrective calculations in the image reconstruction processor 31 that minimize the motion artifacts in the reconstructed image. Alternatively, the amplitude and frequency of breathing may be calculated from an envelope curve of the electrocardiogram (EKG) signal and provided to the image reconstruction processor 31, or the image fusion unit 32. If, in addition, the times of the images made are recorded, then 4D (movie-like) views can be reconstructed from the 3D images. A variety of video displays 33, which may be flat panel displays, may be provided to present images and data for manipulation and analysis.

A patient monitor, or physio-sensor 50, such as described in U.S. Pat. No. 6,221,012, “Transportable Modular Patient Monitor with Data Acquisition Modules”; or as a product, the Infinity Gamma (available from Dräger Medical Deutschland GmBH, Lübeck, Germany), may sense the blood pressure, heart rate, oxygen saturation, and EKG, and the data may be stored in a memory 80, along with image and other data obtained from the various sensors.

An ultrasound device, for instance, the iLook device from Sonosite, http://www.sonosite.com/ and/or the HandyScan from Primedic, may be employed so that the progress of some aspects of the therapy can be followed or controlled, without emitting X-radiation.

The patient monitor 50 and the ultrasound device, for example, may be combined into one unit, as described for instance in U.S. Patent Application 2004/0249279, “Patient Monitor for Processing Signals from an Ultrasound Probe”. In another aspect, the patient monitor, the ultrasound device and a defibrillator (not shown) may be combined into a unit, as described in German patent application 2005P04026, not yet published, Serial No. 102005031642.5.

A trans-esophageal echocardiography (TEE) examination may be performed, and an apparatus (not shown) for performing trans-esophageal echocardiography may be provided. Such a TEE apparatus is known, for example, from U.S. Pat. 6,142,941, “Device for Carrying Out a Transoesophageal Echocardiography and a Cardioversion.”

In another aspect, an ACUNAV catheter 68 from Siemens AG (Erlangen, Germany) can be used. This catheter may advanced through the venous system into the heart, whether manually or using the robot 63, and can be used for generating ultrasound images from the chambers of the heart. Such a robotically controlled catheter and a guide wire may be used to emplace the occluder during therapy.

An image fusion unit 32 or function (e.g., recording, segmenting, superimposing) may combine information from different imaging devices and the motion sensor 15. For example, sonograms can be fused with the X-ray and angiographic images in 2D, 3D or 4D image representations.

A compact blood sugar analysis device, such as Accu-Check from Roche Diagnostics GmbH (Mannheim, Germany) may be used for determining the blood sugar values. In addition, a blood analysis device, such as “Lab on a Chip”, which is being developed by Siemens AG, may be used for determining further blood values or certain genetic or molecular markers (see, for example, WO 00/56922, “Genetic Polymorphism and Polymorphic Pattern for Assessing Disease Status, and Compositions for Use Thereof”, and DE 69919885, “Method for Measuring Cellular Adhesion” for gene tests and tests with molecular markers for stroke). See also, WO 2005/106024, entitled “method and Assembly for DNA Isolation with Dry Reagents” and WO 2005/106023, entitled “PCR Process and Arrangement for DNA Amplification using Dry Reagents”, as examples of devices and methods which may be used. For purposes of illustration in FIG. 1, these patient physiological monitors and analysis devices are shown as the physiological sensor 50. As medical knowledge increases, further test devices and methods may be added to the treatment suite.

A computer device 70 may be a notebook, such as a SIMpad (Siemens AG, Erlangen, Germany), or other processing device with which the demographic, history, diagnosis or therapy data of the patient can be recorded, called up and sent to and from the medical information management system of the hospital. The computer device 70 may be provided with a data interface for retrieving data from an HMO (health maintenance organization), health insurance smart card, or other patient data base, and may be connected to the remainder of the therapy suite by a wired or a wireless connection. A user input device 71, such as a keyboard, computer display device, and mouse, may be provided for manual input and control.

In addition, the examination and therapy actions already performed may be documented, including the medications administered or still to be administered. Some or all of the data may be forwarded to another entity or within a facility by use of an internetworking environment 44 for use in diagnosis, billing and administrative purposes, or for further image processing and storage, using known interfaces such as DICOM (Digital Communications in Medicine) 40 and SOARIAN, or other special purpose or later developed data formatting, storage, and processing techniques. SOARIAN is a web-browser-based information management system for medical use, integrating clinical, financial, image, and patient management functions and facilitating retrieval and storage of patient information and the performance of analytic tasks (available from Siemens Medical Solutions Health Service Corporation, Malvern, Pa.).

The examples of diseases, syndromes, conditions, and the like, and the types of examination and treatment protocols described herein are by way of example, and are not meant to suggest that the method and apparatus is limited to those named, or the equivalents thereof. As the medical arts are continually advancing, the use of the methods and apparatus described herein may be expected to encompass a broader scope in the diagnosis and treatment of patients.

Generally a medical workflow follows hospital routine procedures prior to and subsequent to the performance of diagnostic tests and treatment. The patient will have been admitted to the hospital and assigned to a medical treatment specialty. The patient demographic information may be entered into the hospital information processing system as part of the admitting procedures, and may be updated as needed.

Diagnostic tests and examinations that may have previously been performed and the data obtained including, for example, laboratory tests results, and imaging studies may also be incorporated into the patient record. Such information may include recording of image data and the reconstruction of high-resolution images of the patient using CT and magnetic resonance imaging (MRI), and the production of 3D images and data sets. Such images and data sets of the relevant region of the patient may be further processed so as to segment the images to display specific views of body structures and tissue types and other attributes of the region being studied, such as the atria or ventricles. Such information may be transmitted to the treatment suite and displayed therein so as to provide supporting information for the interventional procedure.

The therapy unit may be operated by the acts summarized below.

In a first workflow example, the patient may be transported to the treatment room and positioned on the treatment table. Such a transportation step may be manual or may use robotic devices. Herein, it would be understood by a person of skill in the art that robotic devices may be generally substituted for a human activity, or used to facilitate a human activity, as such robotic devices are being introduced into the hospital environment. The use of such robotic devices may be presumed as at least an optional part of the workflow, unless specifically excluded.

The patient may be connected to some of all of the instrumentation shown in FIG. 1 so as to provide for vital signs monitoring, synchronization of the bodily respiration or cardiac cycle with the imaging suite, for position tracking of catheters, and the like. The specific procedure may commence by puncturing of a femoral or other vein to introduce a catheter into the patient, and the catheter may be threaded to the heart using, for example, radiography or extracorporeal ultrasound guidance. A diagnosis of the shunt type and severity may be performed by a number of techniques, which may be selected by the medical personnel based on prior initial diagnosis, or on the basis of information acquired while the patient is in the treatment suite.

Such techniques as blood pressure measurement and/or oximetry (% SpO2 partial oxygen saturation), or 3D image reconstruction where images of the heart with and without contrast agent may be compared with one another, or superimposed on one another may be used. High-resolution 3D images and data sets may be obtained with an interventional cardiac angiography device such as an AXIOM Artis dTA DynaCT using EKG synchronization and soft-tissue image processing. The CT-like image data set may be segmented to display selected structures or tissue types in the relevant image region, such as the heart chambers. Additional extracorporeal ultrasound images and/or ICE or TEE images may be obtained.

Where a shunt is found between the atrial chambers, the size of the aperture may be determined directly from the 3D images. Alternatively, for example, an elastic balloon catheter filled with contrast agent may be introduced into the atria and expanded until the opening in the heart is closed. The balloon diameter may be measured in the CT image data.

Based on the visualization of the defect, and the measurement of the size of the shunt opening, if interventional treatment by use of an occluder is indicated, an occluder of the appropriate size and configuration may be selected and introduced into the heart by way of venous access. The occluder may be guided to the heart opening to be closed using 2D fluoroscopic images or images or synthetic locations from other sensors, which may be fused with, or superimposed on, 2D/3D images previously recorded. Subsequently, the positioning of the occluder may be facilitated with other imaging modalities, including ultrasound, or previously recorded data. As image processing and fusion techniques are continuing to be improved, the selection of specific views and processing techniques may vary from those herein described, yet perform a similar or equivalent function.

After disposing the occluder so as to close the opening or shunt, high-resolution CT-like images may again be obtained and reconstructed using the C-arm X-ray device, with or without contrast, so as to monitor the results of the therapy. Such images may be synchronized with the cardiac cycle by using EKG data.

Where the post-intervention outcome is satisfactory, the occluder placed in the shunt may be disconnected from the catheter guide wire, so as to remain in the shunt opening when the catheter is withdrawn from the patient. The catheter and other instruments that have been introduced into the patient may now be removed, and post-intervention procedures, as are known in medical arts, performed prior to moving the patient from the treatment suite to a recovery area. In some instances, an extracorporeal ultrasonic examination may be performed to further document the results of the treatment.

In a second workflow example, the aspects of positioning the patient, connecting appropriate instrumentation, puncturing a femoral vein and introducing a catheter may be performed as described in the first workflow example. The shunt size determination may be made by obtaining 3D CT-like images using the C-arm X-ray device, with and without administration of a contrast agent, and synchronizing the images with the cardiac cycle by the use of an EKG. The image data may be reconstructed using CT-type algorithms, as is known, and the volumetric data segmented so as to isolate such structures as the atria. The shunt size (typically, the diameter) may be measured directly from the segmented 3D images. In an alternative, an elastic balloon may be introduced or floated into the shunt region and the balloon expanded until the opening in the heart has been closed. The diameter of the balloon resulting in closure of the shunt may be measured from the 3D images.

Once the size and type of occluder has been determined using the imaging data, the occluder may be introduced into the patient using a catheter and venous access. The occluder may be guided to the appropriate position so as to close the opening using the integrated imaging suite. Acoustic, fluoroscopic and 3D data may be used as needed. Once the opening has been closed, and the results of the intervention confirmed, the remainder of the procedure may be performed in a manner similar to that described for the first workflow procedure.

In a third example, the aspects of positioning the patient, connecting appropriate instrumentation, puncturing a femoral vein and introducing a catheter may be performed as described in the first or second workflow examples, including the steps of determining the size and type of occluder to be used in the interventional treatment.

The occluder may be located with respect to the patient and with respect to one or more position sensors, which may use acoustic or magnetic sensing so as to locate the position of the occluder. The occluder may be guided to the position of the heart opening to be closed using the position sensors and one or more of the image sensors, and positioned so as to close the opening using one or more of the imaging sensors.

Once the opening has been closed, the remainder of the interventional workflow procedure may be performed in a manner similar to the first and the second workflow examples.

The workflows examples described provide an efficient and rapid procedure for closing diseased openings in a patient heart, and may be selected either pre-interventionally or intra-interventionally, as the circumstances may indicate.

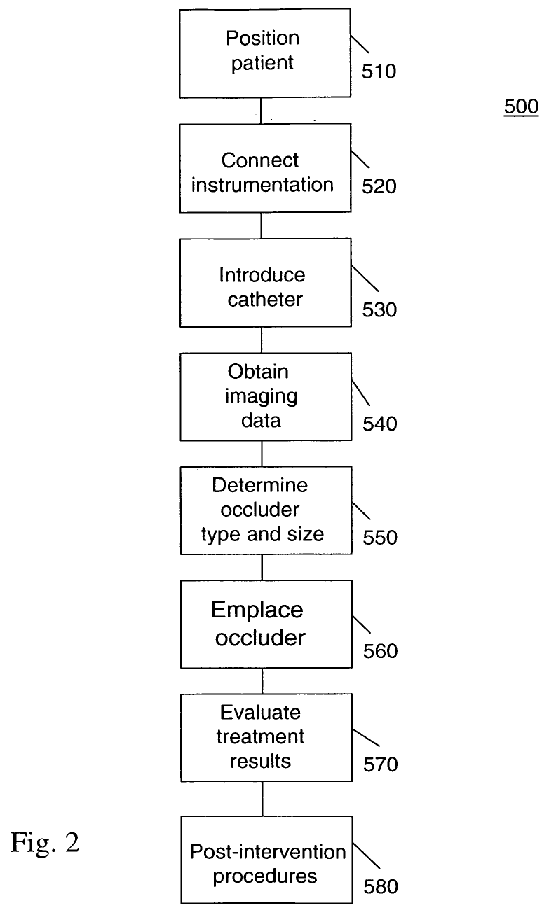

To illustrate a workflow in more detail, a top-level flow chart is shown in FIG. 2. The interventional procedure 500 commences by moving the patient to the treatment equipment suite (step 510), followed by connecting the appropriate monitoring equipment, which may include an EKG, and vital functions monitor, or the like (step 520). A catheter is introduced (step 530) into the patient, and the imaging modality suite used to obtain images (step 540) for use in determining the type and size of occluder to be used (step 550). The occluder may be emplaced in the patient (step 560) using the imaging modality suite, and acoustic or magnetic positioning devices may be used to assist in the navigation of the occluder to the site of the heart opening. After the occluder has been emplaced and the heart opening has been closed, the imaging suite may be used to monitor the results so as to determine if the procedure has been successful (step 570). When the interventional procedure has been completed, the catheter and any other equipment may be either removed from the patient body, or disconnected therefrom, and post-intervention procedures performed (580), which ends with the transfer of the patient from the therapy unit.

While the workflows and methods disclosed herein have been described and shown with reference to particular steps performed in a particular order, it will be understood that these steps may be combined, sub-divided, or reordered to from an equivalent workflow method without departing from the teachings of the present invention. Accordingly, unless specifically indicated herein, the order and grouping of steps is not a limitation of the present invention.

Although only a few exemplary embodiments of this invention have been described in detail above, those skilled in the art will readily appreciate that many modifications are possible in the exemplary embodiments without materially departing from the novel teachings and advantages of the invention. Accordingly, all such modifications are intended to be included within the scope of this invention as set forth in the following claims.

What is claimed is:

1. A method of treating cardiovascular syndromes, the method comprising:
positioning a patient in a therapy unit;
determining a size of an aperture between structures of a heart using a C-arm X-ray device; and
emplacing an occluder in the aperture using a catheter and guide wire.

2. The method of claim 1, wherein the patient is positioned by a robotic device.

3. The method of claim 1, further comprising determining the size of the aperture by measuring a size of a balloon containing radio-opaque material sufficient to close the opening.

4. The method of claim 3, wherein the step of measuring uses images obtained using the C-arm X-ray device.

5. The method of claim 1, further comprising determining the size of the aperture by processing C-arm X-ray data to produce CT-like images and segmenting the CT-like images to isolate a structure having the aperture.

6. The method of claim 5, wherein the CT-like images are synchronized with at least one of a cardiac cycle or a breathing cycle.

7. The method of claim 6, wherein the synchronization with the cardiac cycle is performed using electrocardiogram (EKG) data.

8. The method of claim 1, further comprising introducing the occluder into the heart using a catheter having a guide wire.

9. The method of claim 8, wherein a position of the occluder is determined by extracorporeal acoustic imaging.

10. The method of claim 8, wherein a position of the occluder is determined by a magnetic sensor.

11. A method of treating a patient, the method comprising:
providing a patient treatment suite, the suite further comprising:
a C-arm X-ray device, configured to produce CT-like 3 dimensional (3D) images or voxel data sets; and
a catheter suitable for introducing an occluder into a heart of the patient;
wherein the occluder is selected based on a measurement of a size of an aperture in the heart of the patient.

12. The method of claim 11, wherein act of measuring the size of the aperture uses an extracorporeal imaging modality.

13. The method of claim 12, wherein the extracorporeal imaging modality is one of the C-arm X-ray device or an acoustic imager.

14. The method of claim 12, wherein the size is a dimension of a balloon that occludes the aperture.

15. A workflow for treating a patient having a septum defect, the workflow comprising the steps of:
placing the patient on a patient support in a treatment unit;
inserting a catheter into the patient;
obtaining images of a heart of the patient using a C-arm X-ray device capable of recording data suitable for the production of CT-like images;
measuring a size of an unwanted aperture in the heart;
selecting an occluder based on the measured aperture size;
introducing the occluder into the patient using a catheter having a guide wire, and emplacing the occluder in the aperture;
detaching the occluder from the guide wire; and
removing the catheter from the patient.

16. The workflow of claim 15, wherein the measuring of the sizes further comprises:
using the catheter to position a balloon in the aperture, and inflating the balloon so that the balloon closes the aperture; and
measuring the diameter of the balloon with an imaging modality.

17. The workflow of claim 16, wherein the balloon is inflated with a radio-opaque material.

18. The workflow of claim 17, wherein the imaging modality is a C-arm X-ray device.

19. The workflow of claim 15, wherein the occluder is guided to the aperture using one of the C-arm X-ray device, an acoustic sensor, or a magnetic sensor.

20. The workflow of claim 15, wherein an image time is related to a cardiac cycle by electrocardiogram (EKG) information.

21. A system for treating a heart syndrome, comprising:
a imaging modality, configured to produce CT-like 3 dimensional (3D) images or voxel data sets; and
a catheter capable of introducing an occluder device into a heart of a patient;
wherein the occluder device is selected based on a measurement of a size of an aperture in the heart of the patient from the 3D images or voxel data sets.

22. The system of claim 21, wherein the imaging modality is a C-arm X-ray device.

23. The system of claim 21, wherein the imaging device is an extracorporeal acoustic imager.

24. The system of claim 21, wherein a position of the occluder is determined by extracorporeal acoustic imaging.

25. The system of claim 21, wherein a diagnosis of the syndrome and the introduction of the occluder is performed without moving the patient to another treatment room
